(19) United States
(12) Patent Application Publication
(10) Pub. No.: US 2003/0017524 A1
(43) Pub. Date: Jan. 23, 2003

(54) Streptococcus agalactiae in vitro diagnostic

(76) Inventors: Jane H. Hall, Sunnyvale, CA (US); Kenneth Borchardt, Novato, CA (US); Robert D. Hall, Sunnyvale, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/186,179

(22) Filed: Jun. 26, 2002

Related U.S. Application Data
(60) Provisional application No. 60/301,536, filed on Jun. 27, 2001.

(51) Int. Cl. C12M 1/34

(52) U.S. Cl. 435/29; 435/287.2

(57) ABSTRACT

Provided are devices, for testing a sample for a specific microorganism, including specific embodiments for determining presence of group B streptococcus, comprising: a first reservoir containing a selective broth and a second reservoir containing a selective agar comprising an indicator moiety, the selectivity being for the specific microorganism, and the indicator moiety being associated with a measurable property change in the presence of the specific microorganism or attribute; and a conduit fluidically connecting the two reservoirs, the conduit having a valve, having an open position and a closed position initially positioned in the closed position. The sample is introduced into the first reservoir and the device is incubated at a first specified temperature for a first incubation period. The valve is then opened to introduce the contents of the first reservoir into the second reservoir and the device is then incubated at a specified temperature for a second incubation period.

### REFERENCE TO RELATED APPLICATIONS

This application claims the benefit of U.S. Ser. No. 60/301,536 filed Jun. 27, 2001, which application is fully incorporated herein by reference.

### BACKGROUND OF THE INVENTION

1. Field of the Invention

This application relates to diagnostic devices, and in particular devices used for testing a sample for a specific microorganism including specific embodiments for determining the presence of group B streptococcus.

2. Description of Related Art

Group B or ∃-hemolytic streptococci (GBS) are increasingly appreciated as important human pathogens. GBS cause meningitis, endocarditis, bronchopneumonia, arthritis, peritonitis, wound infections, abscesses, and urinary tract infections in adults. But up to 80% of group B infections occur in neonates (Jelinkova, J. (1977) Current Topics in Microbiology and Immunology: 76:127-165). Studies have evidenced that approximately 30% of pregnant women are colonized by GBS. Despite this high carrier rate, neonatal infection occurs with an incidence of only 0.5% (Lira et al. (1986) J. Clin. Micro.: 23:489-492). Predisposing factors to development of disease are premature birth, prolonged rupture of membranes, overt maternal infection, and deficiency of type specific antibody (Boyer et al. (1986) New England J. Med.: 314:1665-1669).

In addition to the need to identify and sub-type GBS, there is an urgent need to identify means of preventing GBS infections. The need to prevent neonatal GBS infections is most acute. GBS infections now account for over 40% of all neonatal sepsis in the United States resulting in over 12,000 cases and 2,500 deaths of neonates annually (Strickland, et al. (1990) Am. J. Obstet. Gynecol.: 163:4-8). And, pregnancy related morbidity occurs in nearly 50,000 women annually (Baker, C. J. (1989) J. I. D.: 161:917-921). In 1985 the National Academy of Sciences estimated the cost of GBS early onset sepsis and obstetric disease exceeded $500 million and listed GBS the fourth most important cause of preventable infectious mortality in the United States (Strickland et al. (1990) supra). As discussed above, up to 30% of pregnant women have been reported to be colonized with GBS. Despite this high degree of colonization, only a small percentage of women actually give birth to septic infants. But, infected neonates experience a high rate of morbidity (particularly permanent neurological damage) and mortality. A GBS vaccine is presently unavailable.

Early onset GBS sepsis in neonates occurs within hours to days of birth, is associated with vertical transmission from the mother, and has a mortality rate as high as 50-60%. Causative GBS strains are not clearly delineated by serotype. Late onset GBS sepsis occurs days to weeks after birth, is not necessarily associated with vertical transmission, and is associated primarily with strains expressing the type III carbohydrate. Serotype III GBS are often associated with meningitis. The mortality rate from late onset sepsis is approximately 20%, but neurologic sequelae occur in approximately 50% of patients with meningitis.

Therefore, there is a great need to identify compositions and methods to improve ability to detect and diagnose infection by GBS. Methods permitting culturing for serotyping along with diagnostic detection are especially valuable. This need is especially acute for diagnosis of pregnant women. Culture methods for prokaryotes including bacteria are generally known, as are methods of culturing GBS.

Culturing methods are commonly employed to detect microbial pathogens in specimens, whether of human, animal or environmental origin. The following general culturing procedure is commonly used: the target (and other) microbes contained by the specimen are inoculated as part of the specimen into a culture medium, which provides all required nutrients for growth. The specimen may be an untreated natural sample, or it may be pretreated as, for example, by membrane filtration, thus concentrating or amplifying the total microbe content that can be practicably cultured in a laboratory culture setup for a total specimen of a certain size. Typically vaginal presence of GBS is detected in a vaginal fluid sample obtained by a vaginal swab.

The culture medium has the nutrients and other selective chemicals such as antimetabolites or antibiotics, which are selectively active against microbes other than the target microbes. The culture medium is a “general medium”, even with the selective chemicals, in that it supports the growth of both target microbes and related microbes and thus is only partially specific to the target microbes.

The culture medium, which may be a water solution or a water gel, is sterilized to rid it of any contaminating microbes which may be present in the medium and which could, therefore, interfere with the analysis. The culture medium must be refrigerated and packaged in such a way to avoid contamination after manufacture.

After one or more of the culture media are inoculated with the specimen, the inoculated media are incubated under controlled atmospheric conditions. After incubation, the culture media are examined for growth of any microbes. If such growth is observed, a sample thereof is taken for further analysis, because the presence of the target microbe can only be established by isolating it in the pure state, not mixed with other microbes. Once isolated on subsequent culture media, the target microbes are identified by testing for a variety of physical and chemical characteristics. Typically a testing or differential medium provides a selective growth medium for the target microbe and includes a specific moiety that either only the target microbe can metabolize, or of a group of microbes selected for the target microbe only can metabolize in a certain manner. The specific moiety may be a nutrient, and often such a specific moiety is a nutrient that is modified by attaching a sample-altering moiety thereto, thereby converting the nutrient to a nutrient-indicator. When such a nutrient-indicator is employed, a sample-altering moiety is activated to alter the sample only if the specific nutrient is metabolized by the target microbe. The sample-altering moiety can be a material which changes the color of the sample (visible or non-visible) or changes an electrical characteristic of the sample, or alters some other detectable characteristic of the sample. Testing media employing such sample altering moieties and methods of using such media are described by Edberg in U.S. Pat. No. 6,329,166.

If the apparent target microbe growths are not isolated, e.g. by selective culturing prior to employing an indicator medium, false negative tests can result.

For GBS, Todd-Hewitt broth having antibiotic additives is an example of a selective broth medium. An antibiotic that selects for the GBS organisms may be employed. Often gentamycin is employed with Todd-Hewitt broth to reduce enterococcal growth to effect a relative concentration of GBS. Pigmentation testing for GBS organisms such as S. agalactiae has long been known (Durand et al. (1923) Compt. Rend. Acad.: 177:133), pigment formation being associated with the action of bacterial ∃-hemolysin (Lancefield (1933) J. Exp. Med.: 59:459-69). Growing a suspected GBS sample on an agar permitting the GBS to produce a pigment has consequently long been an accepted technique for identification of GBS. Various such agar media are known including: Columbia (Meritt et al. (1976) J. Clin. Micro.: 3:287-90; Haug et al. (1977) Acta Pathol. Microbiol. Scand. Sect. B: 85:286-88; Jokipii et al (1976) J. Clin. Pathol.: 28:736-39); DMS; Islam (Islam et al. (1977) Lancet i: 256-57; Waitkins et al. (1980) J. Clin. Pathol.: 33:302-305; Waitkins et al. (1982) Med. Lab. Sci.: 39:185-88); Granada (de la Rosa et al. (1983) J. Clin. Micro.: 18:779-85; Ros a-Fraille et al. (1999) J. Clin. Micro.: 37:2674-77); pigment (Albers et al. (1983) Am. J. Med. Technol.: 49(11):807-11). An indicator broth for ∃-hemolysis has also been used to determine presence of GBS (Noble et al. (1983) J. Clin. Pathol.: 36:350-52).

There remains a need for a storable portable and integrated test kit for detection by culturing of GBS which is relatively free of the risk of post sample-inoculation contamination, and permits isolation of the selectively cultured GBS from positive tests for subsequent serotyping thereof.

### SUMMARY OF THE INVENTION

An object of the present invention is to provide a test relatively free of the risk of post sample-inoculation contamination.

An object of the present invention is to provide an improved test over currently known tests.

Another object of the present invention is provide a test that requires less time and labor to give a result.

The invention is directed to devices for testing for presence in a sample of a specific microorganism or a specific microorganism attribute. The device comprises: a first reservoir containing a selective broth and a second reservoir containing a selective agar comprising an indicator moiety, the selective broth and the selective agar being selective for the specific microorganism or the specific microorganism attribute, and the indicator moiety being associated with a measurable property change in the presence of the specific microorganism or the specific microorganism attribute; and a conduit fluidically connecting the first reservoir and the second reservoir, the conduit having a valve, the valve having an open position and a closed position the valve initially positioned in the closed position. The sample is introduced into the first reservoir and the device is incubated at a first specified temperature for a first incubation period. The valve is then caused to be in the open position to introduce the selective broth contained in the first reservoir into the second reservoir. The device is then incubated at a second specified temperature for a second incubation period. Presence of the specific microorganism or the specific microorganism attribute is indicated by the measurable property change. In some embodiments, more convenience may be achieved by observing through the pouch wall without opening the test pouch.

In a preferred embodiment the invention is directed to a device for testing for presence in a sample of a specific microorganism or a specific microorganism attribute comprising: (i) a pouch comprising a first reservoir containing a selective broth and a second reservoir containing a selective agar comprising an indicator moiety, the selective broth and the selective agar being selective for the specific microorganism or the specific microorganism attribute, and the indicator moiety being associated with a measurable property change in the presence of the specific microorganism or the specific microorganism attribute; and (ii) a conduit integrated into the pouch, the conduit fluidically connecting the first reservoir and the second reservoir, the conduit having a valve, the valve having an open position and a closed position the valve initially positioned in the closed position. The sample is introduced into the first reservoir and the device is incubated at a first specified temperature for a first incubation period. The valve is then caused to be in the open position to introduce the selective broth contained in the first reservoir into the second reservoir. The device is then incubated at a second specified temperature for a second incubation period. Presence of the specific microorganism or the specific microorganism attribute is indicated by the measurable property change.

In specific embodiments of the preceding embodiments, the invention comprises a device for testing for presence in a sample of a group B streptococcus (∃-hemolytic streptococcus) microorganism. Another specific embodiment detects a prokaryote having the specific attribute of ∃-hemolysis, which can be a streptococcus, or another ∃-hemolytic prokaryote, depending upon the selective additives used for the broth and the agar.

In various pouch embodiments, the invention further comprises a member whereby without opening the pouch, the valve may be moved to the open position by pushing the member. Alternatively the valve in pouch embodiments is: a section of the conduit having a collapsed position corresponding to the closed position, and pushing the member through the section of the conduit obtains the open position; or valve is an impermeable membrane, the impermeable membrane being intact when the valve is in the closed position, and pushing the member punctures the membrane to obtain the open position.

In the various embodiments for culturing prokaryotes, including prokaryotes capable of causing pathology in a immunocompetent or immunocompromised human or animal may be tested according to the invention with the appropriate selective and indicator media. According to the general embodiments of the invention, various gram positive and gram negative prokaryote pathogens, both aerobic and anaerobic can be tested. For example according to the invention gram positive cocci (spheres) including facultative anaerobes of genus Streptococcus and Staphylococcus. Aerobic gram positive rods (bacilli) of genus Escherichia Bacillus, including B. subtilis and B. anthracis, the cause of anthrax may also be tested by culturing according to the various embodiments of the invention. And intracellular prokaryotes may also be cultured for detection, intracellular prokaryotes including both obligate and facultative intracellular prokaryotes, facultative intracellular prokaryotes including Legionella, invasive species including members of genus Listeria, Salmonella and Shigella and the zoonoses, e.g. members of genus Bordatella, Yersinia, Brucella, Borellia and Francisella, including Bordatella pertussis and other strains having as a significant animal host man. In preferred embodiments Group B streptococcus is detected by culturing.

The device is incubated at about 32-40° C. for the first incubation period and at about 32-40° C. for the second incubation period. Preferably the device is incubated at about 34-38 ^{B}C for the first incubation period and at about 34-38° C. for the second incubation period, more preferably at about 35-37° C. for the first incubation period and at about 35-37° C. for the second incubation period. Most preferably the device is incubated at about 37° C. for the first incubation period and at about 37° C. for the second incubation period. The incubation ranges of the device for prokaryotes are: about 2 to 12 hours for the first incubation period and about 4 to 22 hours for the second incubation period; preferably about 4 to 10 hours for the first incubation period and about 6 to 20 hours for the second incubation period; more preferably about 6 hours for the first incubation period and 8 to 10 hours for the second incubation period. Expressed as minimum incubation times for specific embodiments of the present invention, the incubation times are: at least about 2 hours for the first incubation period and at least about 6 hours for the second incubation period; preferably at least about 4 hours for the first incubation period and at least about 8 hours for the second incubation period. Expressing the first incubation period as a minimum incubation time and the second incubation time as a range, the incubation is: at least about 4 hours for the first incubation period and about 8 to 18 hours for the second incubation period.

For various microorganisms an indicator moiety associated with a measurable property change in the presence of the specific microorganism or the specific microorganism attribute is employed, in which the measurable property change may be: color, osmolality, pH or presence of a specific chemical moiety. For example the measurable property change may be appearance of a pigment observable as a color change. In embodiments directed to detection of GBS by culture, a color change to orange indicates presence of group B streptococcus in the sample.

In some embodiments the selective broth comprises a Todd-Hewitt broth that is typically modified with addition of antibiotics for selectivity, and the selective agar typically comprises a Granada agar. Selectivity of broth and/or agar is typically by employing appropriate antibiotic additives to select a specific organism, specific microorganism describing individual strains and species and members of a specified genus or a group of microorganisms having a common attribute. Thus when selecting GBS the selective broth may comprise a Todd-Hewitt broth having appropriate antibiotic additives for selecting GBS and the selective agar comprises a Granada or Islam agar having appropriate antibiotic additives for selecting GBS.

### BRIEF DESCRIPTION OF THE DRAWINGS

The various features and aspects of the instant invention may be more readily understood, in conjunction with the description to follow, by reference to the following drawings:

FIG. 1 depicts an embodiment of the invention as a folded pouch comprising two containers for the selective broth and indicator agar.

FIG. 2 depicts a cross section of the embodiment of the invention of FIG. 1, showing the two containers and a conduit having a valve between the two containers.

FIG. 3A depicts one configuration of an embodiment of the invention having a valve assembly.

FIG. 3B depicts another configuration of an embodiment of the invention having a valve assembly.

### DETAILED DESCRIPTION OF THE PREFERRED EMBODIMENTS

The invention is directed to devices for testing for presence in a sample of a specific microorganism or a specific microorganism attribute. The device comprises: a first reservoir containing a selective broth and a second reservoir containing a selective agar comprising an indicator moiety, the selective broth and the selective agar being selective for the specific microorganism or the specific microorganism attribute, and the indicator moiety being associated with a measurable property change in the presence of the specific microorganism or the specific microorganism attribute; and a conduit fluidically connecting the first reservoir and the second reservoir, the conduit having a valve, the valve having an open position and a closed position the valve initially positioned in the closed position. The sample is introduced into the first reservoir and the device is incubated at a first specified temperature for a first incubation period. The valve is then caused to be in the open position to introduce the selective broth contained in the first reservoir into the second reservoir. The device is then incubated at a second specified temperature for a second incubation period. Presence of the specific microorganism or the specific microorganism attribute is indicated by the measurable property change.

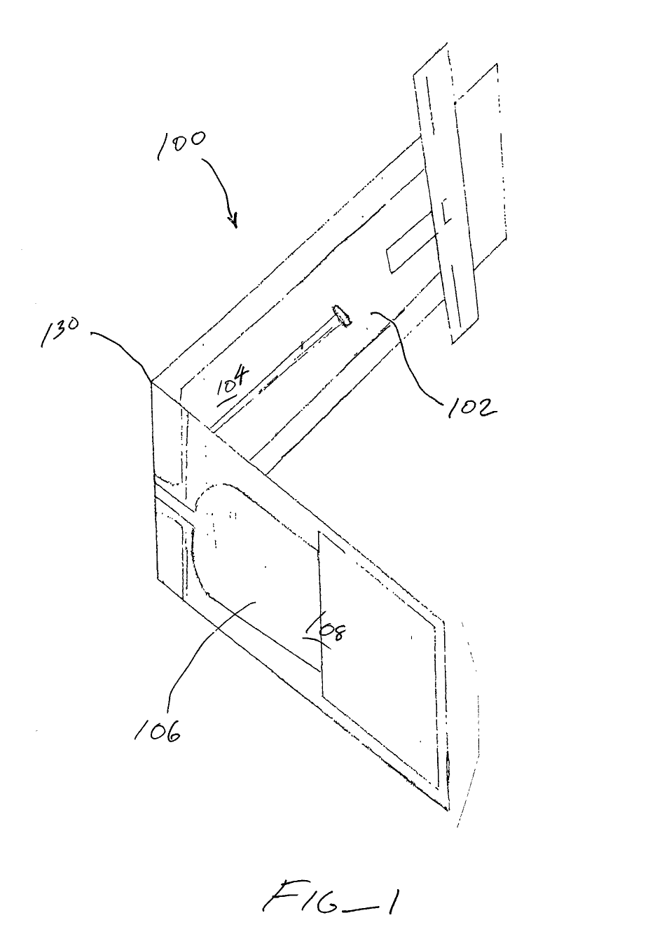

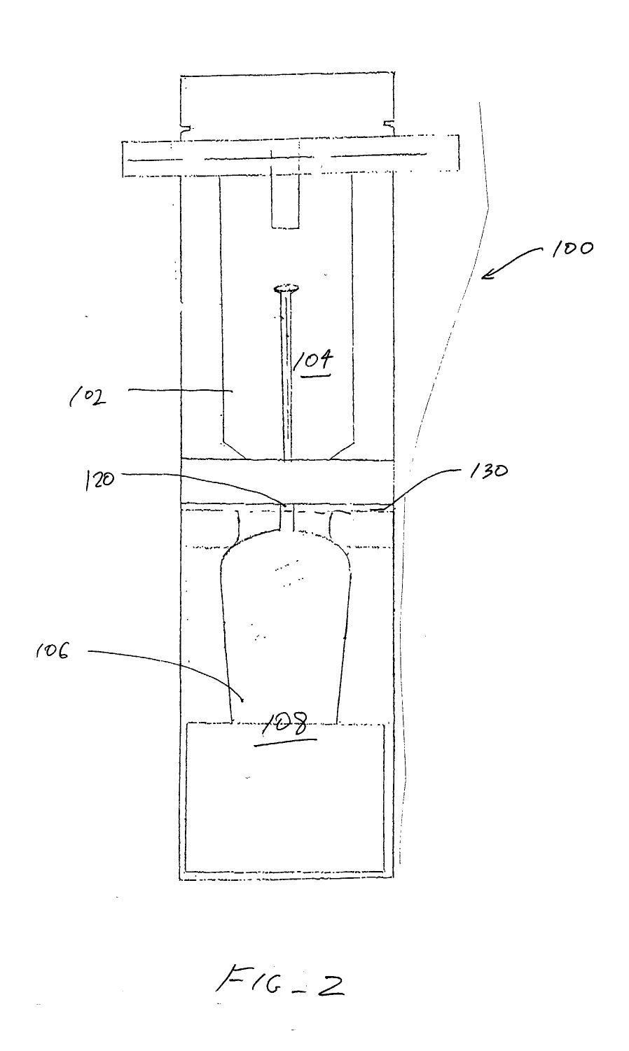

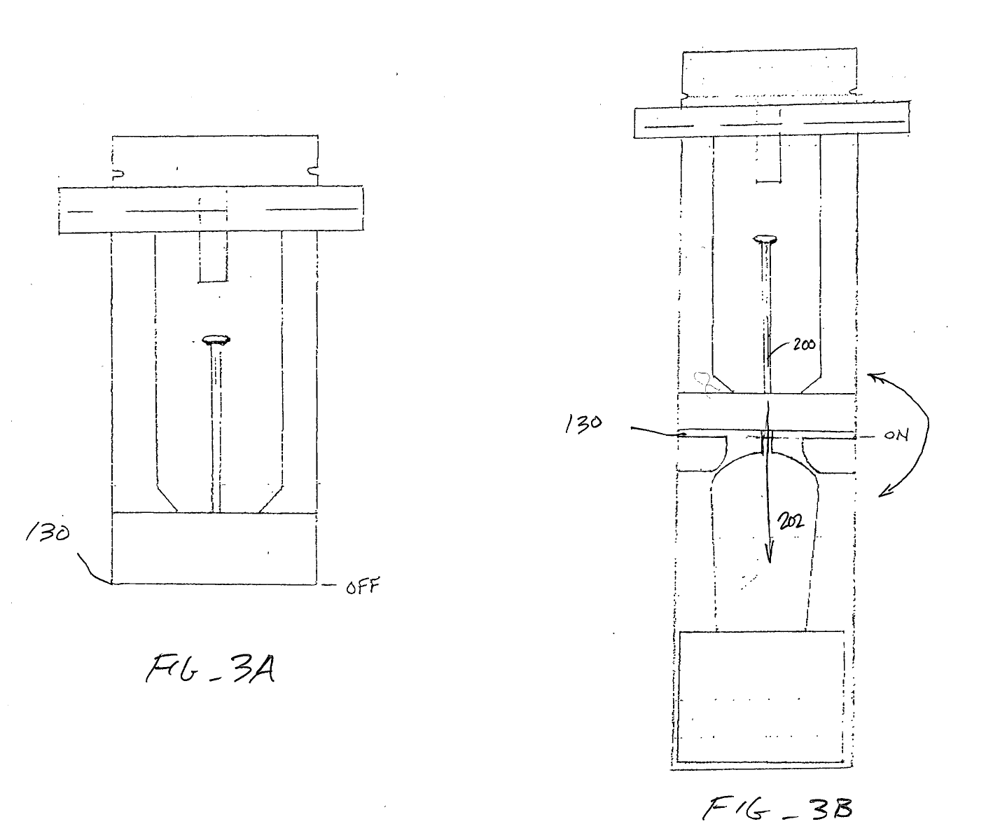

In one embodiment as seen in FIGS. 1 and 2, the invention is directed to a device for testing for presence in a sample of a specific microorganism or a specific microorganism attribute comprising: (i) a pouch 100 comprising a first reservoir 102 containing a selective broth 104 and a second reservoir 106 containing a selective agar 108 comprising an indicator moiety, the selective broth and the selective agar being selective for the specific microorganism or the specific microorganism attribute, and the indicator moiety being associated with a measurable property change in the presence of the specific microorganism or the specific microorganism attribute; and (ii) a conduit 120 integrated into the pouch, the conduit fluidically connecting the first reservoir and the second reservoir, the conduit having a valve 130, the valve 130 having an open position (see FIG. 3A) and a closed position (see FIG. 3B), the valve initially positioned in the closed position. The sample is introduced into the first reservoir 102 and the device is incubated at a first specified temperature for a first incubation period. The valve 130 is then caused to be in the open position to introduce the selective broth contained in the first reservoir 102 into the second reservoir 106. The device is then incubated at a second specified temperature for a second incubation period. Presence of the specific microorganism or the specific microorganism attribute is indicated by the measurable property change.

Referring now to FIGS. 3A and 3B, one specific embodiment of the present invention will now be described. In this embodiment, the pouch 1000 to be used is removed from a refrigerated environment and allowed to equilibrate to room temperature. The pouch 100 may be labeled with any desired test or patient information. The pouch 100 may be stood upright on the valve 130 as in seen in FIG. 3A. The pouch 100 may be opened sufficiently to admit a swab by pulling closure tapes middle tabs apart. A sample or specimen from the patient is obtained. A swab or other equivalent device having the patient sample or specimen is inserted into the first reservoir 102, preferably into the broth 104 to deposit the sample therein. One may squeeze the sample from the swab by gently pressing the swab between the walls of the first reservoir. The swab may then be removed from the first reservoir 102 after having deposited it sample therein. Closing the tape of the pouch 100 without squeezing the liquid or broth 104, the top edge of the pouch may be folded down and rolled. The pouch may then be incubated in a manner according to the present invention.

Referring now to FIG. 3B, the pouch 100 may be moved into this configuration after initial incubation. In this particular embodiment, a probe 200 may be slide through the conduit 120 and into the lower chamber as indicated by arrow 202, without opening the pouch 100. The probe may be fully slid into the chamber. Optionally, the probe may be slid in a manner sufficient to introduce materials from first reservoir 102 into reservoir 106. It should be understood, however, that some embodiments of the present invention may not incorporate a slidable probe as shown herein. In this particular embodiment, the upper portion of the pouch having first reservoir 102 may be rolled to the to the conduit 120 and refastened in this position by closure tabs to hold it so. The pouch 100 may then be placed in an incubator in a manner according to the present invention.

In specific embodiments of the preceding embodiments, the invention comprises a device for testing for presence in a sample of a group B streptococcus (∃-hemolytic streptococcus) microorganism. Another specific embodiment detects a prokaryote having the specific attribute of ∃-hemolysis, which can be a streptococcus, or another ∃-hemolytic prokaryote, depending upon the selective additives used for the broth and the agar.

In various pouch embodiments, the invention further comprises a member whereby without opening the pouch, the valve may be moved to the open position by pushing the member. Alternatively the valve in pouch embodiments is: a section of the conduit having a collapsed position corresponding to the closed position, and pushing the member through the section of the conduit obtains the open position; or valve is an impermeable membrane, the impermeable membrane being intact when the valve is in the closed position, and pushing the member punctures the membrane to obtain the open position.

In the various embodiments for culturing prokaryotes, including prokaryotes capable of causing pathology in a immunocompetent or immunocompromised human or animal may be tested according to the invention with the appropriate selective and indicator media. According to the general embodiments of the invention, various gram positive and gram negative prokaryote pathogens, both aerobic and anaerobic can be tested. For example according to the invention gram positive cocci (spheres) including facultative anaerobes of genus Streptococcus and Staphylococcus. Aerobic gram positive rods (bacilli) of genus Bacillus, including B. subtilis and B. anthracis, the cause of anthrax may also be tested by culturing according to the various embodiments of the invention. And intracellular prokaryotes may also be cultured for detection, intracellular prokaryotes including both obligate and facultative intracellular prokaryotes, facultative intracellular prokaryotes including Legionella, invasive species including members of genus Listeria, Salmonella and Shigella and the zoonoses, e.g. members of genus Bordatella, Yersinia, Brucella, Borellia and Francisella, including Bordatella pertussis and other strains having as a significant animal host man. In preferred embodiments Group B streptococcus is detected by culturing.

The device is incubated at about 32-40° C. for the first incubation period and at about 32-40° C. for the second incubation period. Preferably the device is incubated at about 34-38° C. for the first incubation period and at about 34-38° C. for the second incubation period, more preferably at about 35-37° C. for the first incubation period and at about 35-37° C. for the second incubation period. Most preferably the device is incubated at about 37° C. for the first incubation period and at about 37° C. for the second incubation period. The incubation ranges of the device for prokaryotes are: 2 to 12 hours for the first incubation period and 4 to 22 hours for the second incubation period; preferably 4 to 10 hours for the first incubation period and 6 to 20 hours for the second incubation period; more preferably about 6 hours for the first incubation period and 8 to 10 hours for the second incubation period. Expressed as minimum incubation times, the incubation times are: at least 2 hours for the first incubation period and at least 6 hours for the second incubation period; preferably at least 4 hours for the first incubation period and at least 8 hours for the second incubation period. Expressing the first incubation period as a minimum incubation time and the second incubation time as a range, the incubation is: at least 4 hours for the first incubation period and 8 to 18 hours for the second incubation period.

For various microorganisms an indicator moiety associated with a measurable property change in the presence of the specific microorganism or the specific microorganism attribute is employed, in which the measurable property change may be: color, osmolality, pH or presence of a specific chemical moiety. For example the measurable property change may be appearance of a pigment observable as a color change. In embodiments directed to detection of GBS by culture, a color change to orange indicates presence of group B streptococcus in the sample.

In some embodiments the selective broth comprises a Todd-Hewitt broth that is typically modified with addition of antibiotics for selectivity, and the selective agar typically comprises a Granada agar. Selectivity of broth and/or agar is typically by employing appropriate antibiotic additives to select a specific organism, specific microorganism describing individual strains and species and members of a specified genus or a group of microorganisms having a common attribute. Thus when selecting GBS the selective broth may comprise a Todd-Hewitt broth having appropriate antibiotic additives for selecting GBS and the selective agar comprises a Granada or Islam agar having appropriate antibiotic additives for selecting GBS.

### EXAMPLE 1

### Titrating Gentamycin for GBS Selection

[Islam or Grenada & Todd Hewitt: add gentamycin in amount determined to be below exp MIC for 95% of strains & recapitulate preferred embodiment with explication of broth and media makeup and prep].

### EXAMPLE 2

Salmonella Selenite broth in upper Chamber. Color indicator agar in lower.

### EXAMPLE 3

[Various possibilities exist: Methicillin resistant staph, alpha hemolytic strep] E Coli 0157 buffered lactose broth in upper chamber. Color indicator agar in lower chamber.

Although exemplary embodiments of the instant invention have been described and depicted, it will be apparent to the artisan of ordinary skill that a number of changes, modifications, or alterations to the invention as described herein may be made, none of which depart from the spirit of the instant invention. All such changes, modifications, and alterations should therefore be seen as within the scope of the instant invention.

While the instant invention is disclosed with reference to preferred embodiments detailed above, it is to be understood that these embodiments are intended in an illustrative or exemplary rather than in a limiting sense, as it is contemplated that modifications will readily occur to those skilled in the art, modifications which will be within the spirit of the invention and the scope of the appended claims. All patents, papers, articles, references and books cited herein are incorporated by reference in their entirety.

We claim:

1. A device for testing for presence in a sample of a specific microorganism or a specific microorganism attribute comprising:
a first reservoir containing a selective broth and a second reservoir containing a selective agar comprising an indicator moiety, the selective broth and the selective agar being selective for the specific microorganism or the specific microorganism attribute, and the indicator moiety being associated with a measurable property change in the presence of the specific microorganism or the specific microorganism attribute; and
a conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of the specific microorganism or the specific microorganism attribute by the measurable property change.

2. The device of claim 1, wherein the device is incubated at about 32-40° C. for the first incubation period and at about 32-40° C. for the second incubation period.

3. The device of claim 1, wherein the device is incubated at about 34-38° C. for the first incubation period and at about 34-38° C. for the second incubation period.

4. The device of claim 1, wherein the device is incubated at about 35-37° C. for the first incubation period and at about 35-37° C. for the second incubation period.

5. The device of claim 1, wherein the device is incubated at about 37 ^{B}C for the first incubation period and at about 37° C. for the second incubation period.

6. The device of claim 1, wherein the device is incubated 2 to 12 hours for the first incubation period and 4 to 22 hours for the second incubation period.

7. The device of claim 1, wherein the device is incubated 4 to 10 hours for the first incubation period and 6 to 20 hours for the second incubation period.

8. The device of claim 1, wherein the device is incubated about 6 hours for the first incubation period and 8 to 10 hours for the second incubation period.

9. The device of claim 1, wherein the device is incubated at least 2 hours for the first incubation period and at least 6 hours for the second incubation period.

10. The device of claim 1, wherein the device is incubated at least 4 hours for the first incubation period and 8 to 18 hours for the second incubation period.

11. The device of claim 4, wherein the device is incubated at least 2 hours for the first incubation period and at least 6 hours for the second incubation period.

12. The device of claim 4, wherein the device is incubated at least 4 hours for the first incubation period and 8 to 18 hours for the second incubation period.

13. The device of claim 4, wherein the device is incubated at least 4 hours for the first incubation period and at least 8 hours for the second incubation period.

14. The device of claim 1, wherein the measurable property change is one or more property change selected from the group consisting of color, osmolality, pH and presence of a specific chemical moiety.

15. The device of claim 13, wherein the measurable property change is one or more property change selected from the group consisting of color, osmolality, pH and presence of a specific chemical moiety.

16. The device of claim 1, wherein the selective broth comprises a Todd-Hewitt broth.

17. The device of claim 1, wherein the selective agar comprises a Granada agar.

18. The device of claim 1, wherein the selective agar comprises a Todd-Hewitt broth and the selective broth comprises a Granada agar.

19. The device of claim 1, wherein the selective broth comprises a Todd-Hewitt broth having appropriate antibiotic additives for selecting a specific microorganism and the selective agar comprises a Granada agar having appropriate antibiotic additives for selecting a specific microorganism.

20. The device of claim 13, wherein the selective broth comprises a Todd-Hewitt broth having appropriate antibiotic additives for selecting a specific microorganism and the selective broth comprises a Granada agar having appropriate antibiotic additives for selecting a specific microorganism.

21. The device of claim 1, wherein the specific microorganism is the members of a specified genus or a group of microorganisms having a common attribute.

22. The device of claim 19, wherein both the Todd-Hewitt broth and the Granada agar select for group B streptococcus.

23. The device of claim 20, wherein both the Todd-Hewitt broth and the Granada agar select for group B streptococcus.

24. The device of claim 23, wherein the measurable property change is appearance of a pigment observable as a color change to orange, indicating presence of group B streptococcus in the sample.

25. The device of claim 1 further comprising a probe slidable between the first reservoir and the second reservoir.

26. A device for testing for presence in a sample of a specific microorganism or a specific microorganism attribute comprising:
a first reservoir containing a selective broth, the selective broth being selective for the specific microorganism or the specific microorganism attribute;
a second reservoir containing a selective agar comprising an indicator moiety, the selective agar being selective for the specific microorganism or the specific microorganism attribute, and the indicator moiety being associated with a measurable property change in the presence of the specific microorganism or the specific microorganism attribute; and
a conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of the specific microorganism or the specific microorganism attribute by the measurable property change property change.

27. A device for testing for presence in a sample of a group B streptococcus microorganism comprising:
a first reservoir containing a selective broth and a second reservoir containing a selective agar comprising an indicator moiety, the selective broth and the selective agar being selective for group B streptococcus microorganisms, and the indicator moiety being associated with a measurable property change in the presence of group B streptococcus microorganisms; and
a conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of group B streptococcus by the measurable property change.

28. A device for testing for presence in a sample of a group B streptococcus microorganism comprising:
a first reservoir containing a selective broth, the selective broth being selective for group B streptococcus microorganisms;
a second reservoir containing a selective agar comprising an indicator moiety, the selective agar being selective for group B streptococcus microorganisms, and the indicator moiety being associated with a measurable property change in the presence of group B streptococcus microorganisms; and
a conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of group B streptococcus microorganisms by the measurable property change.

29. A device for testing for presence in a sample of a ∃ hemolytic prokaryote comprising:
a first reservoir containing a selective broth and a second reservoir containing a selective agar comprising an indicator moiety, the selective broth and the selective agar being selective for ∃ hemolytic prokaryotes, and the indicator moiety being associated with a measurable property change in the presence of ∃ hemolytic prokaryotes; and
a conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of ∃ hemolytic prokaryotes by the measurable property change.

30. A device for testing for presence in a sample of a ∃ hemolytic prokaryotes microorganism comprising:
a first reservoir containing a selective broth, the selective broth being selective for ∃ hemolytic prokaryotes;
a second reservoir containing a selective agar comprising an indicator moiety, the selective agar being selective for group ∃ hemolytic prokaryotes, and the indicator moiety being associated with a measurable property change in the presence of group B streptococcus microorganisms; and
a conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of ∃ hemolytic prokaryotes by the measurable property change.

31. A device for testing for presence in a sample of a specific microorganism or a specific microorganism attribute comprising:
a pouch comprising a first reservoir containing a selective broth and a second reservoir containing a selective agar comprising an indicator moiety, the selective broth and the selective agar being selective for the specific microorganism or the specific microorganism attribute, and the indicator moiety being associated with a measurable property change in the presence of the specific microorganism or the specific microorganism attribute; and
a conduit integrated into the pouch, the conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of the specific microorganism or the specific microorganism attribute by the measurable property change property change.

32. The device of claim 31 further comprising a member whereby without opening the pouch, the valve may be moved to the open position by pushing the member.

33. The device of claim 31, wherein the valve is a section of the conduit having a collapsed position corresponding to the closed position, and pushing the member through the section of the conduit obtains the open position.

34. The device of claim 31, wherein the valve is an impermeable membrane, the impermeable membrane being intact when the valve is in the closed position, and pushing the member punctures the membrane to obtain the open position.

35. A device for testing for presence in a sample of a group B streptococcus microorganism comprising:
a pouch comprising a first reservoir containing a selective broth and a second reservoir containing a selective agar comprising an indicator moiety, the selective broth and the selective agar being selective for group B streptococcus microorganisms, and the indicator moiety being associated with a measurable property change in the presence of group B streptococcus microorganisms; and
conduit integrated into the pouch, the conduit fluidically connecting the first reservoir and the second reservoir, the conduit having a valve, the valve having an open position and a closed position the valve initially positioned in the closed position;
wherein introducing the sample into the first reservoir and incubating the device at a first specified temperature for a first incubation period, then causing the valve to be in the open position to introduce the selective broth contained in the first reservoir into the second reservoir and incubating the device at a second specified temperature for a second incubation period indicates presence of group B streptococcus by the measurable property change.
